(19) United States
(12) Patent Application Publication
(10) Pub. No.: US 2017/0296717 A1
(43) Pub. Date: Oct. 19, 2017

(54) Suction tubing in "Y" two-way

(71) Applicant: Jose Gustavo Espino, Alexandria, VA (US)

(72) Inventor: Jose Gustavo Espino, Alexandria, VA (US)

(21) Appl. No.: 15/131,981

(22) Filed: Apr. 18, 2016

(51) Int. Cl. A61M 1/00; A61B 1/00; A61B 1/317; A61M 39/10

(52) U.S. Cl. CPC A61M 1/0086; A61M 39/105; A61B 1/00128; A61B 1/317

(57) ABSTRACT

The suction system in the surgical area is part of the operation room, one of its functions is helps to aid the vision of the surgeon and the surgical team. Currently the operation rooms use a regular suction tubing which is just one female connector to the suction system, a body tube and a female connector to the surgical field.

With the modification and adaptation of the regular suction tubing, we will get the new suction tubing in “Y” two-way for surgery, one unit will do the job of 2 regular suction tubing, which will help in saving time to connect, and avoid changing tubes between instruments during the surgery (uncomfortable), less tangles in the surgical field and it will impact in the environment since we will producing fewer PVC which means less material to be disposed.

### FIELD

This invention relates to the suction system in surgeries that is commonly used in the different Operations Rooms of the hospitals and surgical centers.

### BACKGROUND

This suction system for surgeries in operation rooms consists of a final collector of fluids (canister, machines like the nautilus, etc) where this collector is connected to a regular sterile suction tubing made of PVC, which consists of 3 meters of longitude and serves to connect between the final collector of fluids (canister, nautilus, etc), and the operation field that depending on the type of surgery it could be connected with a cannula, other surgical instruments, and collector bags or scopes. Some types of surgeries need to open 3 o more regular suction tubing in order to connect each instrument; this creates a stress to the surgical team because they need to plug in and take care of each wire, as well as each tube to prevent those from getting tangled, bending, twisting and falling which would lead to contamination.

The principal function of the suction system is to provide clear vision to the surgeon during the surgery, as the suctioning of blood, body fluids, and the suction of liquids used when the wounds are being washed occurs. In addition, this would be used a mean of vision in arthroscopies and cystoscopy.

Currently, the most common use of a minima invasive surgery is the trend like the laparoscopy surgeries and the arthroscopy surgeries. On the latter type of surgery is where they have appeared news instruments to help the surgeon as the cameras, shaver, electrocauteries, which simultaneously need a suction system each one, which involve the use of 2 regular suction tubing at least in each surgery ,some surgeries need 4 regular suction tubing, creating difficulties to connect each suction tubing plus the connection of the cables of the instruments.

### SUMMARY

By providing a new device (suction tubing in “Y” two way for surgeries) which would perform the function of 2 regular suction tubing, there would be multiples benefits, such as:

1—saving time: fewer amounts of tubes being connected to the suction system, as well as, less boxes to open, and less tangling between cables and tubes.

2—saving money: by opening a suction tubing in “Y” two-way for surgeries, it would be cheaper than to open 2 regular suction tubing.

3—environment friendly: all those tubes are disposables, but the length of the suction tubing in “Y” two-way has a much shorter length (<2 meters) than the open 2 regular suction tubing.

We can take as a reference Inova Alexandria hospital, which they reported to have 1000 arthroscopies on 2015. For those surgeries they used 2000 suction tubes (3 meters each one=6000 meters)

If we were to use the suction tubing in “Y” two ways in the same hospital , with the same time and numbers of surgeries, we would just need 1000 suction tubing in “Y” two ways that will only account for 4000 meters of PVC. The difference of 2000 meters a year in just one hospital can make a big difference for the environment. Now just imagine this in the nation wide and world wide.

### BRIEF DESCRIPTION OF THE DRAWING

The following content describes the suction tubing in “Y” two-way where:

FIG. 1: suction tubing in “Y” two-way where we can see a female connector to the suction system (2), a body tube with linear ridges in the surface (1), a connector of three ways in “Y” shape (3), two extremities tubes with linear ridges in the surface (4), and two female connectors to the surgical field (5)

### DETAILED DESCRIPTION

The suction tubing in “Y” two way for surgeries like the regular suction tubing used in the present is made of PVC in its whole.

The suction tubing in “Y” two-way is an improvement of the regular suction tubing.

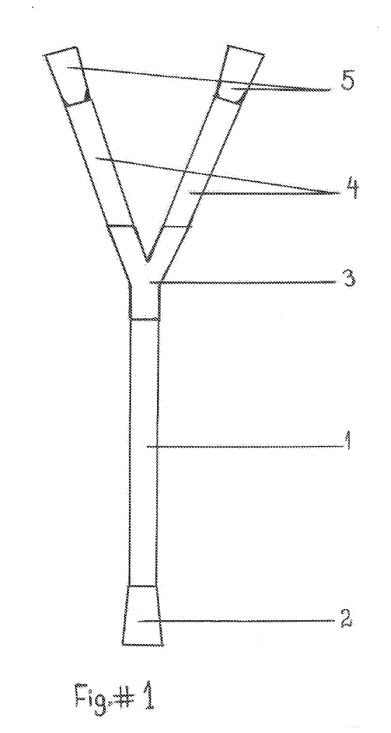

The suction tubing in “Y” two-way illustrated in the FIG. 1: have two female connectors to the surgical field (5), two extremities tubes with linear ridges in the surface (4), a connector in three ways in “Y” shape (3) made of polypropylene or a material stronger that PVC to avoid bending, twisting; a body tube with linear ridges in the surface (1); a female connector to the suction system (2). Unlike the regular suction tubing which is just one female connector to the surgical field, a body tube and a female connector to the suction system.

At the present the most common use of a minima invasive surgery is the trend like the laparoscopy surgeries and the arthroscopy surgeries on the latter type of surgery, where they have appeared more instruments to help the surgeon; such as, the scope, electrocautery, and the shaver. Is in this kind of surgery where the suction tubing in “Y” two way is most useful.

Regarding Arthroscopy surgeries; the Hip, knee, shoulder are the most frequently performed.

To organize one of those types of surgeries (arthroscopies) the surgical tech need to at least open 2 suction tubing, sometimes 3 o 4 tubing in order to make the instruments work appropriately. The connection of all those tubes plus the cables of light, camera, shaver, and electrocautery, make to the surgical team stressed, since they have to prevent each cable and tube from getting tangled, twisted, bended or contaminated.

For this reason, suction tubing in “Y” two-way illustrated FIG. 1, is going to be a great help because on those cases we just need to open one unit (when we need two regular suction tubing) or two units (when we need 4 regular suction tubing) since each unit have 2 female connectors for the surgical field, and one female connector for the suction system of the operation room.

With this suction tubing in “Y” two-way:

1—The nurse or the circulating person will be opening less packages of suction tubing by surgery, less connections to the suction system.
2—It's environment friendly because the regular suction tubing has a length of 3 meters +−5 cm according the different companies. Now if we need 4 tubes, it will be 12 meters of PVC (disposables). With the suction tubing in “Y” two-way which are 4 meters length for unit, for this same surgery we just need 2 units, which will only account for 8 meters of PVC, making a big difference of 4 less meters of PVC. This is a big impact for the environment since producing fewer PVC means that there will be less material to be disposed at the end of each surgery, making a better overall impact.
3—The suction tubing in “Y” two-way can be used for other kind of surgeries, such as, spinal surgeries, bilateral mastectomies, and ACL surgeries.

1- suction tubing in “Y” two-way for surgeries
Characterized by being composed of a body tube with linear ridges in the surface; in one of the ends it has a female connector for the suction system and the other end is connected to the lower part of a connector of three ways in “Y” shape, this connector in “Y” shape in the upper part is connected in each way with extremities tubes with linear ridges in the surface, whose extremities end each one with a female connector for the surgical field.

2- suction tubing in “Y” two-way for surgeries
According to claim 1 characterized by having a connector of three ways in “Y” shaped which in the lower part is connected to the body tube with linear ridges in the surface and in the upper parts connected in each way with extremities tubes with linear ridges in the surface.

3- suction tubing in “Y” two-way for surgeries
According to claim 2 characterized for having extremities tubes with linear ridges in the surface which are connected to the upper part of the connector of three ways in “Y” shape and finished each extremity in a female connector to the surgical field which will be connected to the different surgical instruments.
